(19) United States
(12) Patent Application Publication
(10) Pub. No.: US 2025/0114798 A1
(43) Pub. Date: Apr. 10, 2025

(54) DEVICE AND METHOD FOR THE SEPARATION AND/OR PURIFICATION OF A COMPOUND OF INTEREST

(71) Applicant: QUANTOOM BIOSCIENCES S.A., Nivelles (BE)

(72) Inventors: Anne DE LAMOTTE, Liège (BE); Jérôme LAVAL, Woluwe-Saint-Pierre (BE); Bastien MAIRESSE, Uccle (BE)

(21) Appl. No.: 18/912,389

(22) Filed: Oct. 10, 2024

Related U.S. Application Data
(63) Continuation of application No. PCT/EP2023/059817, filed on Apr. 14, 2023.
(60) Provisional application No. 63/331,531, filed on Apr. 15, 2022.

(30) Foreign Application Priority Data
Apr. 20, 2022 (BE) BE2022/5291

(51) Int. Cl. B03C 1/01; C12N 15/10

(52) U.S. Cl. CPC B03C 1/01; C12N 15/1013; B03C 2201/18; B03C 2201/26

(57) ABSTRACT

The current disclosure relates to a device for separating and/or purifying a compound of interest comprising
a sample plate, said sample plate comprising a base portion and one or more sample holders provided on said base portion for receiving a sample container, said sample container is suitable to hold a sample comprising a liquid holding said compound of interest and magnetic particles;
a magnet unit positioned at each sample holder, said magnet unit is configured to capture or to introduce a movement of the magnetic particles;
   wherein the sample holders are configured to perform a mechanical motion, such that the magnetic particles are mixed with the liquid. Methods for separating and/or purifying a compound of interest are equally disclosed.

### CROSS-REFERENCE

This application is a Continuation of International Application No. PCT/EP2023/059817, filed Apr. 14, 2023, which claims the benefit of U.S. Provisional Application No. 63/331,531 filed Apr. 16, 2022, and Belgium Application No. BE2022/5291, filed Apr. 20, 2022, each of which is incorporated herein by reference in its entirety.

### FIELD OF THE INVENTION

The present disclosure relates to devices and methods for performing a purification step during compound production or the purification of molecules such as nucleic acids. More in particular, devices and methods are disclosed allowing purification by means of magnetic particles.

### BACKGROUND

Magnetic bead separation is a technique that is a commonly used technique at lab scale for the purification of compounds generally in the field of testing or at an early stage of research. In general, magnetic particles are linked to a substance that shows an affinity for the compound that needs to be purified. By applying a magnetic field, the magnetic particles with the bound compound of interest are drawn to the magnet, allowing any remaining liquid to be removed. As such, compounds of interest can be separated and/or purified from their liquid environment.

The methodology of magnetic bead purification is often used in the production of biomolecules such as proteins, peptides, or nucleic acid.

### SUMMARY

The present disclosure and embodiments thereof serve to provide a solution to one or more of disadvantages disclosed below. To this end, the present disclosure relates to an automated device for separating and/or purifying a compound of interest. More specifically, the device comprises a sample plate equipped with sample holders. Sample containers comprising the compound of interest in a liquid medium are positioned inside the sample holders. A magnet unit is positioned at each sample holder. Magnetic particles are introduced into said samples and mixed with the compound of interest. The magnetic particle-bound compound of interest is then subjected to washing and elution steps, inside the device.

In a second aspect, the present disclosure relates to a system for separating and/or purifying a compound of interest. More in particular, the system comprises at least one device for separating and/or purifying the compound of interest. Robotic arms, injectors, and/or pumps are used for dispensing and removing components and liquids in the sample containers.

In a final aspect, the present disclosure relates to a method. More in particular, the method as described herein relates to a method for separating and/or purifying a compound of interest using the device and the system disclosed herein.

### DESCRIPTION OF FIGURES

FIG. 1A and FIG. 1B shows a representation of a device according to preferred embodiments of the disclosure.

FIG. 2A and FIG. 2B presents the principle of capturing magnetic beads using the magnetic unit. In FIG. 2A the beads are free and in FIG. 2B the beads are captured.

FIG. 3 shows a representation of a sample holder according to an embodiment of the disclosure.

FIG. 4 shows a representation of the sample holder according to another embodiment of the disclosure.

FIG. 5 presents a schematic representation of an embodiment of the system, having two adjacent devices.

FIG. 6 presents a schematic representation of an embodiment of the system, having two nested devices.

FIG. 7 schematically presents a robotic arm according to an embodiment of the invention.

FIG. 8 shows a representation of a system according to preferred embodiments of the disclosure.

FIG. 9 shows a representation of a system according to preferred embodiments of the disclosure.

### DETAILED DESCRIPTION

U.S. Pat. No. 10,364,428 relates to methods and kits for post-IVT RNA purification using magnetic beads. U.S. Pat. No. 9,244,069 discloses a sample plate comprising a plurality of wells and the use of a carousel and a magnetic device for magnetic bead separation; the carousel is designed to dispense the magnetic beads in said sample plate. WO2005008219 also discloses the use of carousels and magnetic systems for washing or purification of products. A device that allows for continuous biomolecules purification and that can be scalable in function of the production needs, has not yet been reported.

While the devices known in the art have their value, it would be advantageous to provide separation and/or purification alternatives that are rapid, efficient, provide good yield, are automated, continuous, and scalable. The current disclosure aims to provide a solution to this.

The present disclosure concerns devices and methods for the purification and/or separation of a compound or molecule of interest. The present disclosure also aims to resolve at least some of the problems and disadvantages discussed below. The devices, systems, and methodologies as described herein allow for a rapid, efficient, and highly automated process, while ensuring a good yield of the molecule or compound of interest. The devices and systems as described allows for a (semi-) continuous production of small or medium batches or volumes of the compound of interest. As such, the production is less time consuming and requires a smaller footprint than those devices and systems producing large volumes. These devices, systems, and methods are in particular useful when applied to the field of nucleic acid purification such as RNA or DNA purification.

### Definitions

Unless otherwise defined, all terms used in this disclosure, including technical and scientific terms, have the meaning as commonly understood by one of ordinary skill in the art to which this disclosure belongs. By means of further guidance, term definitions are included to better appreciate the teaching of the present disclosure.

As used herein, the following terms have the following meanings:

“A”, “an”, and “the” as used herein refers to both singular and plural referents unless the context clearly dictates otherwise. By way of example, “a compartment” refers to one or more than one compartment.

“About” as used herein referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less of and from the specified value, in so far such variations are appropriate to perform in the disclosure. However, it is to be understood that the value to which the modifier “about” refers is itself also specifically disclosed.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Furthermore, the terms first, second, third and the like in the description and in the claims, are used for distinguishing between similar elements and not necessarily for describing a sequential or chronological order, unless specified. It is to be understood that the terms so used are interchangeable under appropriate circumstances and that the embodiments described herein are capable of operation in other sequences than described or illustrated herein.

The recitation of numerical ranges by endpoints includes all numbers and fractions subsumed within that range, as well as the recited endpoints.

The expression “% by weight”, “weight percent”, “% wt” or “wt %”, here and throughout the description unless otherwise defined, refers to the relative weight of the respective component based on the overall weight of the formulation.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or >7 etc. of said members, and up to all said members.

As used herein the term “magnetic particle” and variations thereof is intended to denote a particle with a magnetic, e.g., paramagnetic or superparamagnetic, core coated with at least one material having a surface to which a compound of interest can reversibly bind.

Unless otherwise defined, all terms used in this disclosure, including technical and scientific terms, have the meaning as commonly understood by one of ordinary skill in the art to which this disclosure belongs. By means of further guidance, definitions for the terms used in the description are included to better appreciate the teaching of the present disclosure. The terms or definitions used herein are provided solely to aid in the understanding of the disclosure.

Reference throughout this specification to “one embodiment” or “an embodiment” means that a particular feature, structure, or characteristic described in connection with the embodiment is included in at least one embodiment of the present disclosure. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure, and form different embodiments, as would be understood by those in the art. For example, in the following claims, any of the claimed embodiments can be used in any combination.

### Devices and Systems

In a first aspect, the current disclosure relates to a device for separating and/or purifying a compound of interest comprising
a sample plate, said sample plate comprising a base portion and one or more sample holders provided on said base portion for receiving a sample container, said sample container is suitable to hold a liquid sample comprising said compound of interest and magnetic particles;
a magnet unit positioned at each sample holder, said magnet unit is configured to capture or to introduce a movement of the magnetic particles;
wherein the sample holders are configured to perform a mechanical motion, such that the magnetic particles are mixed with the liquid.

The device as disclosed herein allows for separation and/or purification of compounds of interest including biopharmaceutical compounds such as DNA, RNA, modified RNA, polypeptides, proteins, and modified proteins. Advantageously, said device allows automation of the separation and/or purification process while executing the procedures continuously and with high precision without requiring human intervention. The production protocols can be directly implemented on the device disclosed herein without any process scale-up as the device mimics small-scale manual operations executed in a laboratory.

In an embodiment, each sample holder of the device is configured to perform the mechanical motion independently from the remaining sample holders. This allows for the execution of distinct steps of the separation and/or purification process in each sample holder. In other embodiments, the sample holders are performing simultaneous motions.

In an embodiment, the mechanical motioning of the sample holders is driven by a motor unit or electromagnetic unit. In a preferred embodiment, an electric motor is used. In another embodiment, a plurality of electromagnets is powered in sequence to generate the motion of the sample holder. In another embodiment, the mechanical motioning of the sample holders is driven by a shaker unit. The mechanical motion of the sample holders can be a rotation around an axis of said sample holder or a shaking or a vibrating motion. The mechanical motion of the sample holders can be activated and deactivated. The purpose of the mechanical motioning includes but is not limited to, homogenization of the mixture formed by the sample and the buffers for separation and/or purification, and/or facilitation of the contact between the magnetic particles and the sample.

In an embodiment, the sample holders can be positioned on top of the base portion or are positioned in pockets or recesses of said base portion.

In an embodiment, the magnet unit of each sample holder comprises a permanent magnet, a temporary magnet or an electromagnet, preferably a permanent magnet. When an electromagnet is used, the magnetic field can be quickly changed by controlling the amount of electric current. In another embodiment each magnet unit comprises an array of magnets where each magnet can be a permanent magnet, a temporary magnet or an electromagnet.

In a preferred embodiment, the magnet unit is positioned along a side portion of said sample holder and extends above said sample holder. In another embodiment, the magnet unit is positioned around the sample holder.

In another embodiment, the sample holder is equipped with at least two, three, four, five, six, seven, eight, nine, or ten magnet units. In an embodiment, said magnet units are positioned along different side portions of said sample holder. Alternatively, all the magnets are positioned along the same side portion of said sample holder.

In an embodiment, the magnet unit is the same length as the sample holder, or of a smaller length. In a particularly preferred embodiment, the magnet unit is rod-like and at least ⅕th of the length of the magnet unit extends above the sample holder. In another embodiment at least ¼, ⅓, ½, ⅖, 2/4, ⅔, 2/1, ⅗, ¾, 3/2, 3/1, ⅘, 4/3, 4/2, 4/1, 5/4, 5/3, 5/2 or 5/1 of the length of the magnet unit extends above the sample holder. In a preferred embodiment, the magnet unit has a height that is proportional to the height of the level of the liquid in the sample container. In a preferred embodiment, the height and thickness of said magnet unit are directly proportional to the sample container surface, in order to ensure a sufficiently large magnetic field for capturing magnetic particles. In another embodiment, the magnet unit extends at least the entire length of the sample container, preferably extending under the sample container.

In yet another embodiment the magnet is bar, horseshoe, disc, sphere, cylinder, or ring-shaped.

In an embodiment, the magnet unit is arranged in a housing, which is preferably open at the side facing the sample holder. Alternatively, the housing is positioned around the sample holder and contains multiple magnets. In another embodiment, the magnet unit is ring-shaped and fully surrounds the sample holder.

The housing can be fabricated from any suitable material known in the art, such as but not limited to polymers, thermoplastics, metals or metals alloys.

The magnet unit may be fixed or movable in position. For instance, the magnet unit may perform vertical or lateral movements. These movements can influence whether or not a the magnetic particles will be attracted to the magnet. If positioned too far, the magnetic particles will remain in the liquid.

In an embodiment, an adaptor is present in the sample holder, to adjust the size of said sample holder. The use of the adaptors allows for the device to be used with sample containers of various shapes and sizes. The adaptors can be made of any material suitable in the art such as PC (polycarbonate), PP (polypropylene), PAI (polyamide-imide) (e.g. Torlon), PI (polyimide) (e.g. Tecasint), PPS (polyphenylsulfide) (e.g. Tecatron), PPSU (polyphenylsulfone) (e.g. Tecason P), PSU (Polysulfone) (e.g. Tecason S), PEI (polyetherimid) (e.g. Tecapei), glass (e.g. borosilicate glass), technical ceramics (e.g. FRIDURIT®), Polyaryletherketone (e.g., Polyetheretherketon (PEEK)), thermoplastics (e.g. DuraForm® Pa or DuraForm® GF), metal or metal alloy.

The sample plate of the device disclosed herein is configured to rotate around an axis, preferably the central axis of said base portion. In a preferred embodiment, the sample plate is configured to rotate clockwise and counterclockwise. In another embodiment, the sample plate is configured to perform a linear movement. The rotation of the sample plate is driven by a motor unit, preferably an electric motor. Rotation of the sample plate allows the delivery of the sample container positioned in the sample holder, to different fixed dispensers, where a component or a liquid can be added or removed from the sample container.

The base portion of said sample plate can be rectangular, polygonal, circular, ellipsoidal, or annular. In an embodiment, the sample plate is circular and has a diameter of between 20 and 50 cm, more preferably between 20 and 40 cm, such as 35 cm or 30 cm. In another embodiment, the sample plate is rectangular. The sample plate can be fabricated from any suitable material known in the art such as polymers, metal, metal alloys, resins, or any nonmagnetic or paramagnetic material. Polymers include but are not limited to polystyrene, PVC, Perspex, or Lucite.

In an embodiment, the sample plate comprises a plurality of sample holders, and said sample holders are positioned at regular intervals along the circumference of said base portion. In another embodiment, said plurality of sample holders are positioned at irregular intervals along the circumference of said base portion. Alternatively, the sample holders are positioned along the sides of said base portion.

In one embodiment, the sample plate can comprise one sample holder or a plurality of sample holders. The sample plate can comprise between 1 and 100, more preferably between 1 and 50 sample holders, more preferably between 1 and 20 sample holders, more preferably between 1 and 16 sample holders, more preferably 12 sample holders. For example, a sample plate can comprise at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 sample holders.

In an embodiment, the sample holder has a shape that accommodates the shape of the sample container. In a preferred embodiment, the cross-section of the sample holder is hexagonal. It would be obvious, however, to one skilled in the art that the cross-section of the sample holder can have any shape, as described above, that can accommodate the shape of the sample container.

In some embodiments, the sample holder is removable from the sample plate. In some embodiments, the sample holder is a fixed part of the sample plate.

In an embodiment, the sample holders may comprise identification (ID) means, for identifying a sample container when being present in said sample holder. The ID means may comprise an RFID tag, a smart label, or a reader for reading an RFID tag or smart label. Smart labels include, but are not limited to QR codes and bar codes.

In another aspect, the present disclosure is also directed to a system comprising one or more devices as previously disclosed and at least one handling apparatus configured for dispensing and/or removing a component or a liquid from a sample container present in a sample holder of said device. Said particle component can be a magnetic particle and said liquid can be deionized water, purified water, a buffered solution, a washing buffer, an elution buffer, a reagent, or a combination thereof, or a waste product. It should be apparent that said liquid is not limited to these.

The handling apparatus may comprise any means suitable in the art, such as an injector, a pump, or a robotic arm, for instance provided with one or more nozzles, needles, and/or tips. Any injector, pump, and robotic arm that is known in the art and is capable of dispensing and/or removing a component or a liquid can be used with the system as disclosed herein. Non-limiting examples of handling apparatuses are syringe pumps, vacuum pumps, peristaltic pumps, centrifugal pumps, or a combination thereof. In another embodiment, the robotic arm handles the sample containers, moving for instance the sample containers from an upstream processing unit to the device. In yet another embodiment the robotic arm moves the sample containers from the device to a downstream processing unit.

Alternatively, the dispensing and/or removing of components is done by the handling apparatus directly via the tubing they are provided with.

In an embodiment, the injectors and pumps are fixed dispensers placed in the vicinity of said sample plate and the sample container is brought to them by the rotation of the sample plate. In an embodiment, the robotic arm can move along three separate axes and is able to access the sample container independent of the rotation of the sample plate. In another embodiment, the robotic arm is positioned on the sample plate or in the center of said sample plate.

In an embodiment, the system as disclosed herein comprises at least one robotic arm. In another embodiment, the system comprises one or more injectors. In yet another embodiment the system comprises one or more pumps. In yet another embodiment, the system comprises a robotic arm and one or more injectors. In yet another embodiment, the system comprises a robotic arm and one or more pumps. In a preferred embodiment, the system comprises a robotic arm, one or more injectors, and one or more pumps.

The one or more handling apparatuses of the system are connected to one or more reagent storage, waste vessels, and/or harvest vessels. Any reagent storage, waste vessels and harvest vessels known in the art can be used in conjunction with the system. Non-limiting examples include bags, vials, tubes, bottles, jars or barrels.

In an embodiment, the one or more handling apparatuses of the system are controlled by motor units, preferably electric motors, and are configured to perform movements. In some embodiments of the system, said movement is a vertical, horizontal, centrifugal, or a 3D movement. In a preferred embodiment, the one or more handling apparatus are operatively coupled to at least one computer processor for controlling said handling apparatus.

In an embodiment of the system, the one or more handling apparatuses are positioned along the periphery of said device. In other embodiments, the injectors and pumps are positioned along the periphery of said device while the robotic arm is positioned in the centrum of the device.

The system may further comprise a control system arranged and adapted to control the dispensing and/or removing of a component or liquid by said handling apparatus. Based on the input information, such as sample ID and sensor data, and predefined algorithms, the control system regulates the performance of the one or more handling apparatuses.

In an embodiment, the system as disclosed herein comprises a plurality of devices as described above. In another embodiment, the system comprises two devices as described above. In a further embodiment said devices are positioned adjacent to each other. Alternatively, said devices are in a nested configuration. A multidevice system allows for the performance of multiple processing steps concomitant such that different stages of the separating and/or purifying can be performed at the same time. Moreover, a multidevice system allows for the continuous operation of said system and for processing of a high number of samples.

The system may comprise at least one harvest vessel, for harvesting a compound of interest. In an embodiment, the system may comprise multiple harvest vessels. In another embodiment, the harvest vessel is positioned in an aperture of the sample plate of the device. In yet another embodiment, the harvest vessel is positioned in the system along the vicinity of the device. In yet another embodiment all the devices are provided with at least a harvest vessel. In yet another embodiment one device is provided with a harvest vessel while the other devices have no harvest vessel.

The system comprises one or more sample containers. Said sample containers are positioned in the sample holders of the device.

In an embodiment, the sample containers are disposable. In another embodiment, said containers can be reused multiple times.

In some embodiments, a material of the harvest vessel and/or sample container comprises a material that is resistant to e.g. cleaning procedures (chemically resistant), extreme temperatures (e.g. denaturation of nucleic acids), extreme pH values (sanitization of the reactor with bases and acids, e.g. with NaOH), mechanical forces (e.g. frictions caused by magnetic particles), and/or corrosion. In additional embodiments, the material of the harvest vessel and/or sample container comprises a material of proper light permeation (transparent, translucent, or opaque) to a corresponding purpose. In some embodiments, the material of the harvest vessel and/or sample container comprises a material of proper gas permeation to a corresponding purpose. In further embodiments, the materials of the harvest vessel and/or sample container should be temperature conductive at working temperatures between 37° C. and 65° C. (e.g. W/(mK) values of at least 10, preferably at least 15). In some embodiments, the inner surface of the harvest vessel and/or sample container comprises a surface material that does not release unwanted compounds that may contaminate the end product. In further embodiments, the materials of the harvest vessel and/or sample container and/or the inner surface thereof are PC (polycarbonate), PP (polypropylene), PAI (polyamide-imide) (e.g. Torlon), PI (polyimide) (e.g. Tecasint), PPS (polyphenylsulfide) (e.g. Tecatron), PPSU (polyphenylsulfone) (e.g. Tecason P), PSU (Polysulfone) (e.g. Tecason S), PEI (polyetherimid) (e.g. Tecapei), glass (e.g. borosilicate glass), technical ceramics (e.g. FRIDURIT®), Polyaryletherketone (e.g., Polyetheretherketon (PEEK)), thermoplastics (e.g. DuraForm® Pa or DuraForm® GF), all of which being chemically resistant, pH resistant, and temperature resistant. In additional embodiments, the materials of the harvest vessel and/or sample container comprise a material for a single-use including, but not limited to, polyethylene terephthalate and other polyethylenes, polyvinyl acetate, polyvinyl chloride. In some embodiments, the materials of the harvest vessel and/or sample container comprise a material having resistance to sterilization process including steam treatment or ethylene oxide (EtO) exposure/gamma irradiation even before adding any reaction-related reagents. In some embodiments, the materials of the harvest vessel and/or sample container provide protection from light (if needed) for medium contained in the harvest vessel and/or sample container. In some embodiments, the harvest vessel and/or sample container are made of any nonmagnetic or paramagnetic material known in the art.

In some embodiments, the harvest vessel and/or sample container are pie wedge shaped, regular or irregular polygon shaped, concave polygon shaped, convex polygon shaped, trigon shaped, quadrilateral polygon shaped, pentagon shaped, hexagon shaped, equilateral polygon shaped, equiangular polygon shaped, heptagon shaped, octagon shaped, nonagon shaped, decagon shaped, hendecagon shaped, dodecagon shaped, tridecagon shaped, tetradecagon shaped, pendedecagon shaped, hexdecagon shaped, heptdecagon shaped, octdecagon shaped, enneadecagon shaped, icosagon shaped, n-gon shaped, or elliptic shaped, preferably circular shaped.

In some embodiments, the harvest vessel and/or sample container comprise a volume of at least about 0.1 ml, about 0.3 ml, about 0.5 ml, about 1 ml, about 1.5 ml, about 2 ml, about 2.5 ml, about 3 ml, about 4 ml, about 5 ml, about 6 ml, about 7 ml, about 8 ml, about 9 ml, about 10 ml, about 12 ml, about 15 ml, about 17 ml, about 20 ml, about 25 ml, about 30 ml, about 35 ml, about 40 ml, about 45 ml, about 50 ml, about 55 ml, about 60 ml, about 65 ml, about 70 ml, about 75 ml, about 80 ml, about 85 ml, about 90 ml, about 95 ml, or about 100 ml. In some embodiments, the harvest vessel and/or sample container comprise a volume of not more than about 100 ml, not more than about 95 ml, not more than about 90 ml, not more than about 85 ml, not more than about 80 ml, not more than about 75 ml, not more than about 70 ml, not more than about 65 ml, not more than about 60 ml, not more than about 55 ml, not more than about 50 ml, not more than about 45 ml, not more than about 40 ml, not more than about 35 ml, not more than about 30 ml, not more than about 25 ml, not more than about 20 ml, not more than about 15 ml, not more than about 10 ml, not more than about 9 ml, not more than about 8 ml, not more than about 7 ml, not more than about 6 ml, not more than about 5 ml, not more than about 4 ml, not more than about 3 ml, not more than about 2 ml, not more than about 1 ml, not more than about 0.5 ml, not more than about 0.3 ml, or not more than about 0.1 ml. In some embodiments, the harvest vessel and/or sample container comprises a volume of between about 1 ml to about 100 ml, between about 10 ml to 90 ml, between about 15 ml to about 80 ml, between about 20 ml to about 70 ml, between about 25 ml to about 60 ml or between about 30 ml to about 50 ml.

In some embodiments, the harvest vessel and/or sample container comprises a volume of at least about 150 ml, about 200 ml, about 250 ml, about 300 ml, about 350 ml, about 400 ml, about 450 ml, about 500 ml, about 550 ml, about 600 ml, about 650 ml, about 700 ml, about 750 ml, about 800 ml, about 850 ml, about 900 ml, about 950 ml, about 1000 ml, about 2000 ml, about 3000 ml, about 4000 ml, about 5000 ml, about 6000 ml, about 7000 ml, about 8000 ml, about 9000 ml, about 10000 ml, about 15000 ml, about 20000 ml, about 25000 ml, about 30000 ml, about 35000 ml, about 40000 ml, about 45000 ml, or about 50000 ml. In some embodiments, the harvest vessel and/or sample container comprises a volume of not more than about 50000 ml, not more than about 45000 ml, not more than about 40000 ml, not more than about 35000 ml, not more than about 30000 ml, not more than about 25000 ml, not more than about 20000 ml, not more than about 15000 ml, not more than about 10000 ml, not more than about 9000 ml, not more than about 8000 ml, not more than about 7000 ml, not more than about 6000 ml, not more than about 5000 ml, not more than about 4000 ml, not more than about 3000 ml, not more than about 2000 ml, not more than about 1000 ml, not more than about 950 ml, not more than about 900 ml, not more than about 850 ml, not more than about 800 ml, not more than about 750 ml, not more than about 700 ml, not more than about 650 ml, not more than about 600 ml, not more than about 550 ml, not more than about 500 ml, not more than about 450 ml, not more than about 400 ml, not more than about 350 ml, not more than about 300 ml, not more than about 250 ml, not more than about 200 ml, not more than about 150 ml. In some embodiments, the harvest vessel and/or sample container comprise a volume of between about 150 ml to about 50000 ml, between about 200 ml to 45000 ml, between about 250 ml to about 40000 ml, between about 300 ml to about 35000 ml, between about 350 ml to about 30000 ml, between about 400 ml to about 25000 ml, between about 450 ml to about 20000 ml, between about 500 ml to about 15000 ml. between about 550 ml to about 10000 ml, between about 600 ml to about 9000 ml, between about 650 ml to 8000 ml, between about 700 ml to about 7000 ml, between about 750 ml to about 6000 ml, between about 800 ml to about 5000 ml, between about 850 ml to about 4000 ml, between about 900 ml to about 3000 ml, between about 950 ml to about 2000 ml or between about 1000 ml to about 1500 ml.

Preferably, the sample containers are configured to contain a volume of 0.3 to 100 mL, more preferably from 0.3 to 20 mL, more preferably from 1 to 10 mL, and more preferably from 1 to 50 mL.

In some embodiments, the harvest vessel and/or sample container comprise a cover or lid, to prevent unwanted components from entering said harvest vessel and/or sample container (for example, RNases, microbial contamination or other degrading compounds or organisms) and from shielding the content of the harvest vessel and/or sample container from the outer environment. In some embodiments, the harvest vessel and/or sample container comprise the lid to limit exchange with the environment. In some embodiments, the lid of the harvest vessel and/or sample container is removable. In some embodiments, the lid of the harvest vessel and/or sample container is not removable. Alternatively, the harvest vessel and/or sample container are uncovered.

In some embodiments, the lid can prevent excessive water evaporation and loss of other critical volatile components. In some embodiments, the lid can prevent oxidation of the reagents or any components. In some embodiments, the lid can provide with protection from light (if needed). In some embodiments, the lid prevents contamination from any other potential chemical compound.

In some embodiments, the lid comprises at least one opening for filling, draining and sampling. In some embodiments, the at least one opening is positioned on the top of the lid.

In an embodiment the sample containers comprise a liquid holding said compound of interest and magnetic particles.

In an embodiment, said liquid results from an IVT reaction. In another or further embodiment, said liquid contains DNA, RNA, modified RNA, polypeptides, proteins, and/or modified proteins. In yet another embodiment said liquid contains at least one reagent. In yet another embodiment said liquid contains one or more reagents used for IVT. In yet another embodiment, said liquid comprises impurities. In a further embodiment said impurities are nucleotides, enzymes, proteins, proteins, DNA templates, dsRNA, or any other by-products of IVT known in the art.

The magnetic particles can have any size suitable for binding nucleic acid, including commercially available sizes, such as a diameter ranging from about 0.3 μm to about 10 μm in diameter, e.g., about 0.3, 0.5, 1, 2, 3, 4, 5, 6, 7, 8, 9, or 10 μm in diameter, including all ranges and subranges therebetween.

In an embodiment, said magnetic particles are carboxyl coated paramagnetic particles, silica-based paramagnetic particles, or combinations thereof. Silica-based magnetic particles can comprise, in some embodiments, a paramagnetic core coated with siliceous oxide, thus providing a hydrous siliceous oxide adsorptive surface to which nucleic acid can bind (e.g., a surface comprising silanol groups). The magnetic particles can, in additional embodiments, be surface-modified to produce functionalized surfaces, such as weakly or strongly positively charged, weakly or strongly negatively charged, or hydrophobic surfaces, to name a few. Alternatively, the magnetic particles styrene divinylbenzene particles, polymethacrylate particles, cross-linked agarose particles, or allyl dextran with N-N-bis acrylamide particles. It will be obvious for one skilled in the art that any material that is suitable for binding nucleic acids, proteins, or other biomolecules, may be used with the device or system as disclosed herein.

The magnetic particles of the system may be dispensed to the sample containers by one of said handling apparatuses.

The compound of interest is able to bind to said magnetic particles by means of an affinity binding. In an embodiment, the compound of interest binds to the magnetic particle in the presence of a binding buffer, preferably a binding buffer containing a chaotropic agent. In the presence of the chaotropic agent, the compound of interest reversibly binds to the magnetic particle.

In another embodiment, the magnetic particle is coated with a ligand that interacts with the compound of interest.

The compound of interest to be purified using the system disclosed herein may be a nucleic acid, a peptide or a protein. By preference, the compound of interest is DNA or RNA, preferably RNA, more preferably mRNA. The term RNA or RNA molecules encompasses long-chain RNA, coding RNA, non-coding RNA, long non-coding RNA, single stranded RNA (ssRNA), double stranded RNA (dsRNA), linear RNA (linRNA), circular RNA (circRNA), messenger RNA (mRNA), self-amplifying mRNA (SAM), Trans amplifying mRNA, RNA oligonucleotides, antisense oligonucleotides, small interfering RNA (SIRNA), small hairpin RNA (shRNA), antisense RNA (asRNA), CRISPR/Cas9 guide RNAs, riboswitches, immunostimulating RNA (isRNA), ribozymes, aptamers, ribosomal RNA (rRNA), transfer RNA (tRNA), viral RNA (vRNA), retroviral RNA or replicon RNA, small nuclear RNA (snRNA), small nucleolar RNA (snoRNA), microRNA (miRNA), and a Piwi-interacting RNA (piRNA). In some embodiments, said RNA comprises modified RNA molecules. In some embodiments, the modification of RNA molecule comprises chemical modifications comprising backbone modifications as well as sugar modifications or base modifications. In this context, a modified RNA molecule as defined herein comprises analogues/modifications, e.g. nucleotide backbone modifications, sugar modifications or base modifications. A backbone modification in connection with the present disclosure is a modification, in which phosphates of the backbone of the nucleotides contained in an RNA molecule are chemically modified. A sugar modification in connection with the present disclosure is a chemical modification of the sugar of the nucleotides of the RNA molecule. Furthermore, a base modification in connection with the present disclosure is a chemical modification of the base moiety of the nucleotides of the RNA molecule. In this context, nucleotide analogues or modifications are selected from nucleotide analogues, which are applicable for transcription and/or translation. In further embodiments, the modified RNA comprises nucleoside modifications selected from 6-aza-cytidine, 2-thio-cytidine, α-thio-cytidine, pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, 5-methyl-uridine, pyrrolo-cytidine, inosine, α-thio-guanosine, 6-methyl-guanosine, 5-methyl-cytdine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, α-thio-adenosine, 8-azido-adenosine, 7-deaza-adenosine.

The system as disclosed herein is used in an embodiment in a production process of RNA. In an embodiment, said system is used in an in vitro transcription (IVT) process or downstream of said IVT process. An IVT reaction is typically comprised of the steps of:
mixing a DNA template with nucleotides, RNA polymerase and other reagents; and
incubating the mixture at a defined temperature for a defined duration during which:
the RNA polymerase ‘reads’ the DNA template and catalyzes the synthesis of the corresponding RNA molecule.
the RNA molecule is provided with a capping structure at its 5′ end by either:
co-translational capping (whereby specific reagents are introduced into the reaction mix at the start of the reaction); or
post-transcriptional capping (=transformation of the starting nucleotide in the already formed RNA molecule to a capping structure by enzymatic action).

Preferably, the system as disclosed herein is used for purifying an mRNA molecule from an IVT process, or for purifying during pre-and/or post-capping. In an embodiment, when the mRNA molecule is co-transcriptional capped, said mRNA is purified after the capping. In another embodiment, when the mRNA is post-transcriptionally capped, the purification step is done prior to capping. In yet another or further embodiment, purification is done post capping. In yet another embodiment, a purification step is done before capping and a second purification step is done post capping. The IVT resulting product contains besides the desired mRNA product, an array of reaction by-products such as salts, nucleotides, enzymes, proteins, DNA templates, or dsRNA. These can interfere with the capping process, reduce the transaction efficiency and overall purity of the final product. Enzymatic capping immediately following IVT, without intermediate treatment of the reaction product, produces reduced amounts or approaching 0% capped mRNA molecules. The system disclosed herein is designed to perform mRNA purification with high precision, in an automated manner under GMP-compliant conditions, and is adaptable to perform the purification upstream and/or downstream capping. The system allows for a continuous production of small or medium volumes of the compound of interest.

Alternatively, the system as disclosed herein is used in an embodiment in DNA synthesis. More in particular, the system may be used for downstream processing of DNA after synthesis, for instance by using a thermocycling synthesis method. A non-limiting example is the use of the system in conjunction with the DNA-preparation method for plasmid amplification. It will however be apparent to the skilled person that any DNA synthesis method known in the art can be used in conjunction with the system as disclosed herein.

The system as disclosed herein can be arranged in a cabinet, preferably with a unit for laminar flow generation. In an embodiment, the cabinet is designed to allow the provision of filtered, sterile air to be circulated within the units. Air filtering means may include for instance a HVAC system with HEPA filters.

The housing of the cabinet may be made of any material suitable in the art such as metal alloy, metal, or plastic. In one embodiment, a cabinet is made from a material comprising aluminum or stainless steel. In a specifically preferred embodiment, said cabinet is made of a material comprising stainless steel.

Preferably, the system and the cabinet are designed and operated that they only require limited handling of the operator. This is to avoid contamination and disturbance of the process conditions. If irregularities are observed, the operator can manipulate the process via one or more control devices present inside or outside the cabinet. These control devices control (parts of) the process taking place in the cabinet. The cabinet may be coupled to one or more control devices that are configured to perform multivariate analysis, automatically control the operation of the processes, and optionally, communicate with components remotely (using, for example, network protocols) in order to control operation in the unit(s).

Each cabinet is preferably mobile and provided with transportation means. Transportation means can include any means suitable in the art, both manually and/or electronically controlled, and include but are not limited to wheels, tracks or rolls.

### Methods

In a final aspect, the disclosure relates to a method for separating and/or purifying a compound of interest, comprising at least the following steps:
(a) Providing one or more sample containers comprising a compound of interest and magnetic particles in a liquid medium, wherein each sample container is positioned in a sample holder;
(b) Allowing said compound of interest to mix with said magnetic particles by means of a mechanical motion of said sample holder;
(c) Capturing or introducing a movement of the magnetic particles towards a magnet unit present in the vicinity of said sample holder, thereby causing a separation of the liquid and the magnetic particles;
(d) Removing said liquid.

In an embodiment, after step (d) a liquid is added to said magnetic beads, and steps (b) to (d) are repeated. In a further embodiment, said steps (b) to (d) are repeated at least 2 to 10 times, 2 to 8 times, 2 to 7 times, 2 to 6 times, 2 to 5 times, 2 to 4 times, or 2 to 3 times, preferably at least 2 to 5 times. Alternatively, steps (b) to (d) are repeated at least 3 to 10 times, 4 to 10 times, 5 to 10 times, 6 to 10 times, 7 to 10 times, 8 to 10 times, or 9 to 10 times.

In some embodiments, the composition of the liquids added in between the reiteration of steps (b) to (d) is different. In other embodiments, the addition of liquids having different compositions alternates with the addition of liquids having the same composition.

In some embodiments, the liquid can be an alcohol, a binding buffer, a wash buffer, or an elution buffer.

Binding buffers mediate the reversible binding between the compound of interest and the magnetic particles. In a further embodiment said binding buffer can comprise a chaotropic agent, alcohol, a PEG, a salt, or a mixture thereof. Said chaotropic agent can be chosen from guanidine salts, such as hydrochloride (GuHCl) and guanidium thiocyanate (GuSCN); lithium salts, such as lithium acetate and lithium perchlorate; or sodium salts such as NaCl and combinations thereof. In an embodiment, said binding buffers are devoid of chaotropic agents.

The alcohol can be chosen from isopropanol, ethanol, methanol, butanol, and combinations thereof. In an embodiment, said alcohol is present at a concentration of 10% to 50%, from 10% to 40%, from 10% to 30%, from 10% to 20, from 15% to 20% v/v, including all ranges and subranges therebetween.

In an embodiment, said binding buffer may comprise PEG, either as alternative to the alcohol or in combination with said alcohol. The concentration of PEG in the binding buffer can range from 10% to 40%, from 20 to 40%, from 20% to 35%, from 20% to 30% or from 25% to 35%,, including all ranges and subranges therebetween. In an embodiment, 30% PEG is used. In an embodiment, said PEG used in the binding buffer is chosen from PEG 600, PEG 1000, PEG 2000, PEG 3000, PEG 4000, PEG 6000, PEG 8000, PEG 10.000, PEG 20.000. In an embodiment, used PEG is PEG 8000.

The at least one salt can be present in the binding buffer in a concentration ranging from 0.1 M to 5 M, for example, from 0.1 to 4 M, from 0.1 M to 3 M, from 0.1 M to 2 M, from 0.1 M to 1 M, from 0.5 to 1 M, from 0.5 to 2 M, from 1 M to 2 M and from 2 M and 3 M and from 3 M to 5 M, including all ranges and subranges therebetween. According to various embodiments, the at least one salt can be sodium chloride (NaCl).

In an embodiment, the binding buffer comprises Tris-HCl, NaCl, EDTA, and ethanol.

In embodiments, said binding buffer can have a pH ranging from 5 to 10, such as from 5 to 9, from 5.5 to 8.5, from 6 to 8, or from 6.4 to 7.5 and all ranges and subranges therein between. In embodiments, said binding buffer comprise of at least one first alcohol and/or PEG, at least one salt and at least one optional chelating agent such as EDTA.

The magnetic particle can be present in the binding buffer in a concentration ranging, for instance, from about 0.1 μg/μl to about 60 μg/μl, such as from about 0.5 μg/μl to about 60 μg/μl, from about 0.75 μg/μl to about 55 μg/μl, from about 1 μg/μl to about 50 μg/μl, from about 2 μg/μl to about 45 μg/μl, from about 3 μg/μl to about 40 μg/μl, from about 4 μg/μl to about 35 μg/μl, from about 5 μg/μl to about 30 μg/μl, from about 6 μg/μl to about 25 μg/μl, from about 7 μg/μl to about 20 μg/μl, from about 8 μg/μl to about 15 μg/μl, or from about 9 μg/μl to about 10 μg/μl, including all ranges and subranges therebetween. By way of non-limiting embodiment, the at least one magnetic particle may be chosen from Qbeads and may be present in the binding buffer B1 in a concentration ranging from about 0.5 μg/μl to about 5 μg/μl. In alternative embodiments, the at least one magnetic particle may be chosen from Grace beads and may be present in the binding buffer B1 in a concentration ranging from about 2 μg/μl to about 60 μg/μl.

In embodiments of the methods as taught herein, a volumetric ratio between the sample and the binding buffer can range, for example, from 1:1 to 1:3, such as from 1:1 to 1:1.5, or from 1:1.5 to about 1:2.5, including all ranges and subranges therebetween. Incubation time period for the mixed solution comprising sample comprising at least one nucleic acid of interest, binding buffer and silica-based magnetic particles can range from 0.1 minute to 30 minutes, from 0.1 minutes to 25 minutes, from 0.1 minutes to 20 min, from 0.1 to 10 minutes, or from 0.1 to 5 minutes, from 0.1 to 2 minutes including all ranges and subranges therebetween.

In an embodiment, after removing the binding buffer, one or more wash buffers are added and steps (b) to (d) are repeated. A wash buffer can comprise, for example, at least one alcohol and optionally at least one salt. The modified magnetic particles can be rinsed once or multiple times with the wash buffer, and any additional washing can employ the same or different compositions, concentrations, and/or volumetric amounts. According to various embodiments, the at least one alcohol can be chosen from isopropanol, methanol, ethanol, butanol, and combinations thereof. According to various embodiments, the at least one salt can be chosen from ammonium sulfate ((NH₄)₂SO₄), ammonium acetate (NH₄Ac), lithium acetate (LiAc), potassium acetate (KAc), sodium acetate (NaAc), sodium chloride (NaCl), and combinations thereof.

In embodiments of the methods as taught herein, said wash buffer comprises at least a second alcohol in a concentration ranging, from 50% to 100% by volume/volume (v/v), from 55% to 95%, from 60% to 85%, or from 60% to 80% by v/v, including all ranges and subranges therebetween. In embodiments, the second alcohol can be chosen from isopropanol, methanol, ethanol, butanol, and combinations thereof. The second alcohol in the wash buffer can be the same or different from the first alcohol in the binding buffer. In some embodiments, the second alcohol can be ethanol. In non-limiting embodiments, the wash buffer optionally has at least one salt. The optional salt, if present, in the binding buffer can is in a concentration ranging from 0.1 M to 5 M, for example, from 0.3 M to 4 M, from 0.1 M to 3 M, from 0.1 M to 2 M, from 0.1 M to 1 M, and from 1 M to 2 M, including all ranges and subranges therebetween. According to various embodiments, the salt in the wash buffer can be sodium chloride (NaCl). In some embodiments, the modified magnetic particles can be washed one or more time with at least one of said wash buffer. For example, said modified magnetic particles may be washed once, twice, or more with the wash buffer with intervals of separation of the modified magnetic particles by using a magnet in between the washes.

After the addition and removal of the wash buffer, the modified magnetic particles with the compound of interest reversibly bound to the surface are substantially free of contaminants such as salts, proteins, enzymes, etc. According to various embodiments, the modified magnetic particles thus produced can then be incubated with one or more elution buffers to release the bound compound of interest and separate it from the magnetic particles.

According to various embodiments, the elution buffer is a low conductivity solution wherein the conductivity of the buffer ranges from 0.001 to 40 mS/cm, more preferably from 0.01 to 40 mS/cm, from 0.1 to 40 mS/cm, from 0.5 to 40 mS/cm, more preferably from 0.5 to 30 mS/cm, from 0.5 to 20 mS/cm, from 0.5 to 10 mS/cm, including all ranges and subranges therebetween.

In another or further embodiment, said elution buffer comprises a salt concentration of between 0.01 and 50 mM, more preferably between 0.1 to 40 mM, more preferably between 0.1 and 30 mM, more preferably between 0.1 and 20 mM.

Possible salts include sodium citrate, sodium chloride, sodium phosphate, potassium chloride, potassium phosphate and combinations thereof.

The pH of the elution buffer can range, for example, from 5 to about 10, such as from 5.5 to about 9, from 6 to 8, or from 6.4 to about 7.5, including all ranges and subranges therebetween.

For example, in some cases elution buffer can comprise water; in others water and EDTA, or only Tris, or Tris and EDTA, or Sodium citrate, or phosphate buffer, or Phosphate-buffered saline (PBS). The concentration of the sodium citrate, if used as elution buffer can range from 0.5 mM to 10 mM, for example from 0.6 mM to 5 mM, from 1 mM to 2 mM, including all ranges and subranges therebetween. The pH of the Sodium citrate, if used as elution buffer can range from pH 5.4 to 7.5, from pH 6 to pH 7, from pH 6 to 6 pH 6.5, including all ranges and subranges therebetween. According to non-limiting embodiments, the elution buffer can comprise water or 10 mM Tris-HCl, 1 mM EDTA, pH 7.4, or 10 mM Tris-HCl, pH 7.4, or 1 mM citrate Na, pH 6.4.

In embodiments, said elution buffer is devoid of toxic chaotropic agents such as guanidine salts (guanidinium thiocyanate or guanidine thiocyanate), iodide, perchlorate and trichloroacetate, preferably guanidine salts.

In embodiments, at least one modified particle incubated with the elution buffer for a time period ranging from 30 seconds to 30 minutes, from 1 minute to 20 minutes, from 1 minute to 10 minutes including all the ranges and subranges therebetween.

Hence, also disclosed herein is a method for nucleic acid purification comprising:
a) combining a sample comprising at least one nucleic acid of interest in a binding buffer having a pH ranging from 5 to 10 with silica-based magnetic particles to form a solution;
wherein the binding buffer comprises at least one salt, present in a concentration ranging from 0.1 M to 5 M, at least one first alcohol present in a concentration between 10% to 50% v/v and/or polyethylene glycol (PEG) in a concentration range of 10% to 40% (v/v)
b) incubating the solution for a time period sufficient to reversibly bind the at least one nucleic acid to the magnetic particles to form modified magnetic particles;
c) separating the modified magnetic particles from the combined solution by applying a magnetic field;
d) washing the at least one modified magnetic particle with at least one wash buffer comprising at least one second alcohol at a concentration of between 60% to 100%,
e) combining the modified magnetic particle with an elution buffer in order to allow the elution of said nucleic acid of interest from said magnetic particle, wherein said elution buffer has a pH ranging from about 5 to 10 and wherein said elution buffer has a conductivity of between 0.001 and 40 mS/cm or wherein the total salt concentration is from 0 to 50 mM.

Once eluted in a suitable buffer, the compound of interest, can be transferred and collected in the harvest vessel.

In some embodiments of the method, the sample holder with a sample container rotates between or after a capturing method step to at least one subsequent position. The sample holders with containers are positioned on a base portion of a sample plate and said sample plate is able to rotate. Rotation of the sample plate allows the delivery of the sample container to different dispensers such as injectors or pumps, where a component or a liquid can be added or removed from the sample container.

In other embodiments of the method, the sample plate rotates between or after a capturing method step, thereby moving the sample holder with a sample container to a subsequent position.

In some embodiments of the method, the magnetic particles are added to the sample container by means of an injector, a pump, or a robotic arm provided with one or more nozzles, needles, and/or tips, as previously described. In further embodiments, the addition and/or removal of a liquid occurs by means of one or more injectors, pumps, or robotic arms provided with one or more nozzles, needles, and/or tips, as previously described.

In an embodiment, the mechanical motion of the sample holder that allows the compound of interest to mix with the magnetic particles is shaking or agitating. The shaking or agitating of the sample holder causes the motion of the magnetic particles which become suspended in the liquid medium and come in contact with the compound of interest.

The magnet unit comprises a permanent magnet in an embodiment of the method disclosed herein. The mechanical motion prevents capturing or introducing a movement of the magnetic particles towards said magnet unit.

In a particularly preferred embodiment, the magnetic particles used in the method disclosed herein are silica based magnetic particles, as previously described.

The final step of the method is the elution of the compound of interest from said magnetic particles by means of the addition of an elution buffer to said sample container. In an embodiment, upon adding said elution buffer, the sample holder is subjected to a mechanical motioning, thereby allowing mixing of said magnetic particles with the elution buffer. Once the motioning stops, the magnetic particles move towards said magnet unit, thereby causing a separation of the elution buffer comprising the compound of interest and the magnetic particles. In a further embodiment, the elution buffer comprising the compound of interest is removed from said sample container and stored in a harvest vessel.

In some embodiments of the method described herein, steps a to d are repeated 2 times, 3 times, 4 times, 5 times, 6 times, 7 times, 8 times, 9 times or 10 times, preferably 2 times. Subjecting the compound of interest to several rounds of binding, washing and elution maximizes the yield of the purified compound of interest?

The compound of interest, separated and/or purified with the method disclosed herein is a nucleic acid, a peptide or a protein, as previously disclosed. In a preferred embodiment, said compound is DNA or RNA, preferably RNA, more preferably mRNA or saRNA.

In an embodiment, the method disclosed herein is a step in the production process of RNA. In an embodiment, the method is used in an in vitro transcription (IVT) process or downstream of said IVT process. In a preferred embodiment, said method is used for purifying an mRNA molecule from an IVT process, or for a purification step during pre-and/or post-capping as previously described.

The method as disclosed herein can be executed by means of a device or system according to embodiments previously described. In an embodiment, the method is semi-or fully automated. The method is rapid and efficient while reproducing small-scale repetitive manual procedures. Said method is compliant with GMP conditions, is not prone to human error, and delivers a product of high purity. In addition, the method allows for (semi)continuous production of a compound of interest, as it allows the continuous production of standardized small volumes of a compound.

The present disclosure will now be further exemplified with reference to the following examples. The present disclosure is in no way limited to the given examples or to the embodiments presented in the figures.

### FIGURES

The present disclosure is in no way limited to the embodiments described in the examples and/or shown in the figures. On the contrary, methods according to the present disclosure may be realized in many different ways without departing from the scope of the disclosure.

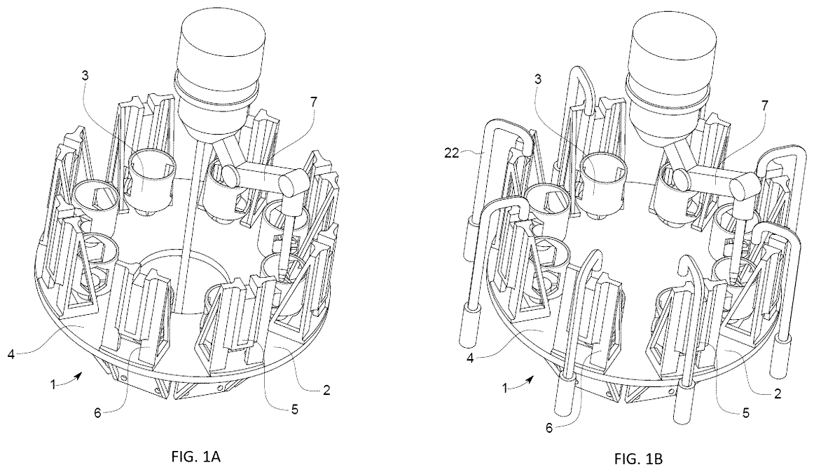

A device (1) for separating and/or purifying a compound of interest according to an embodiment of the current specification is illustrated in FIG. 1 wherein the device is in the form of a carousel. According to the particular embodiment illustrated in FIG. 1., the device (1) comprises a sample plate (2) equipped with sample holders (3) disposed on the base portion (4) of the sample plate (2). In the embodiment of FIG. 1 the sample plate (2) comprises eight sample holders (3). It will however be apparent to the skilled person that this number is flexible. Hence, other embodiments are contemplated wherein a different number of sample holders (3) are provided. Sample containers (8) comprising the compound of interest in a liquid medium are positioned inside the sample holders (3).

A magnet unit (5) is positioned at each sample holder (3). The magnet unit (5) as shown in the embodiment of FIG. 1 is arranged in a housing (6) that is open at the side facing the sample holder (3). In some embodiments, the magnet unit (5) comprises a permanent magnet, in other embodiments comprises a temporary magnet or an electromagnet.

The sample holders (3) can perform mechanical motions that in some embodiments are rotations around their axis and in other embodiments are shaking motions. The sample holders (3) may move independently from one another or may synchronize their motions. In the example shown in FIG. 1 the motion is driven by a motor unit or a shaker unit. The sample plate (2) is configured to rotate around an axis clockwise and counterclockwise.

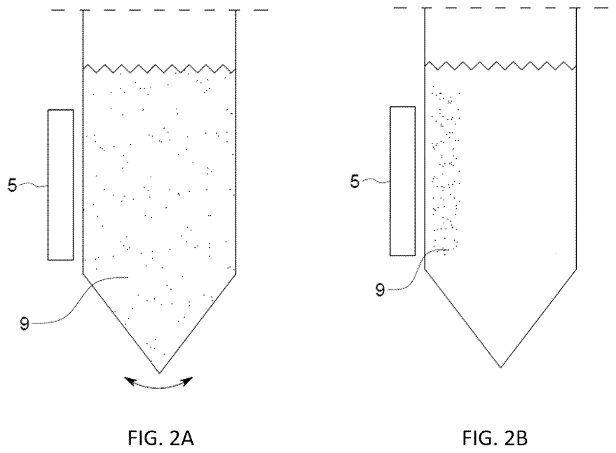

In the embodiment shown in FIG. 1A, a robotic arm (7) is positioned in the centrum of the sample plate (2). In the embodiment of FIG. 2B,, the robotic arm (7) is positioned outside the sample plate (2) or the device (1). The robotic arm (7), equipped with nozzles (17), needles and/or tips, is configured for dispensing and/or removing components and/or liquids from the sample containers (8).

In an embodiment, the compound of interest to be purified is an mRNA produced by an IVT reaction. A sample containing said mRNA suspended in a reaction medium is dispensed in a sample container (8) by a robotic arm (7). Magnetic particles (9) are also dispensed in the sample container (8) by the robotic arm (7).

Capturing of the magnetic particles (9) is shown in FIG. 2. The magnet unit (5) is configured to attract and capture said particles (9) residing in the sample container (8). The motion of the sample holder (3) drives the captured or free status of the silica beads (9). When the sample holder (3) is in motion, the magnetic particles (9) are free to move in the liquid and interact with the mRNA molecules (FIG. 2A). The mRNA molecules reversibly bind to said silica beads (9). When the sample holder (3) stops its motion, the silica beads (9) become captured (FIG. 2B). In an embodiment where an electromagnet is used, the captured or free status of the magnetic particles is controlled by activating and deactivating the electric current.

While the magnetic particles (9) are captured inside the sample container (8), the liquid content of said container can be removed or replaced without losing the magnetic particles (9) or mRNA bound to them. Dispensing and removing of liquids in the sample container is performed by one or more handling apparatus such as robotic arms (7), injectors (20, 21, 22, 27), or pumps (19). The robotic arm (7) can move at any random location and can access any sample container (8). The injectors (20, 21, 22, 27) or pumps (19) are located at fixed positions and the sample containers (8) are delivered to them by the rotation of the sample plate (2). The robotic arm (8) and the other handling apparatuses are connected to one or more reagent storage (29), waste vessels, and harvest vessels (28).

In an embodiment, the device is designed for executing one or more steps of mRNA purification: binding of the mRNA to magnetic particles in the presence of a chaotropic agent, washing of the silica beads bound mRNA, elution of the mRNA, and collection of the purified mRNA in a harvest vessel. This allows for performing multiple automated and precise washing steps, without losing the mRNA. The device is configured to allow repetitive separation and/or purification of mRNA and can be operated continuously. In a preferred embodiment, the magnetic particles are silica-based magnetic beads.

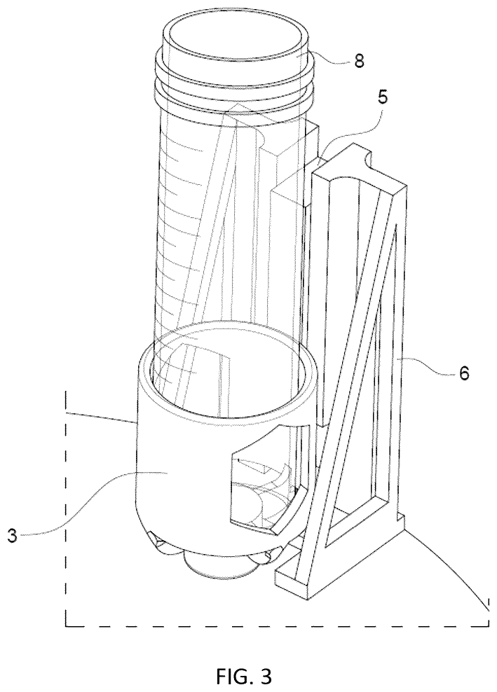

In FIG. 3 an embodiment of the sample holder (3) with a sample container (8) having a capacity of 50 ml, is shown. Said sample container (8) if fitted directly in the sample holder (3). It will be apparent that also other sample containers can be used in the context of the current disclosure.

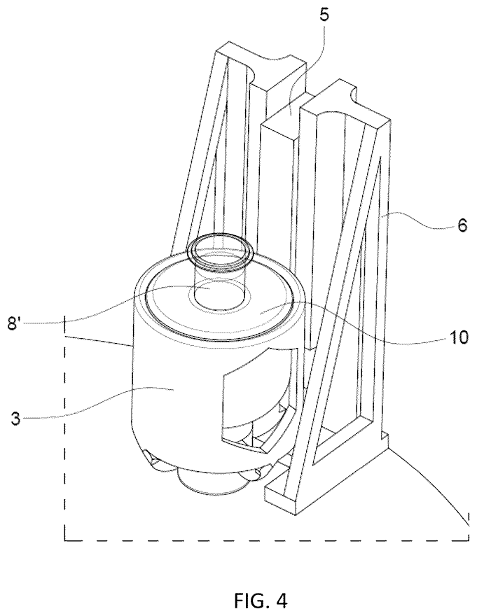

FIG. 4 shows an embodiment of a sample holder (3) with a sample container (8′) having a capacity of 2 ml. Said sample container (8′) is fitted in an adaptor (10) that adjusts the sample holder (3) for the size of the sample container (8′). In other embodiments, sample containers (8′) of other sizes and volumes are used.

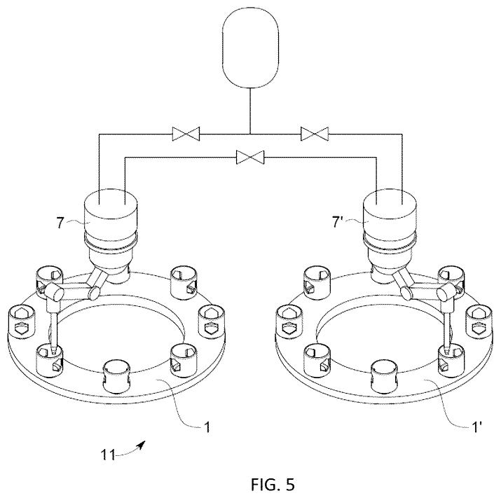

A particular embodiment of the system (11) is shown in FIG. 5, wherein said system (11) comprises two devices (1 and 1′) and two robotic arms (7 and 7′) are provided. In this embodiment, said devices are positioned adjacent to each other. This configuration allows for a higher capacity of production while maintaining the footprint of the devices to a minimum. Alternatively, this configuration can be used for different purification processes. For instance, a first device (1) can be used for pre-capping purification, whereas a second device (1′) can be used in a post-capping process. The capping reaction can take place in the final position of the first device (1), or in an intermediate vessel positioned downstream of the first device (1) and upstream of the second device (1′).

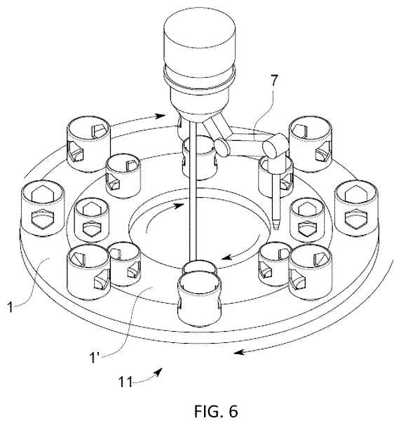

An alternative embodiment is provided in FIG. 6, wherein two devices (1 and 1′) are in a nested configuration and a single robotic arm (7) is provided.

A multidevice system as depicted in FIG. 5 and FIG. 6, allows for the performance of multiple steps concomitant such that different stages of the separating and/or purifying can be performed at the same time.

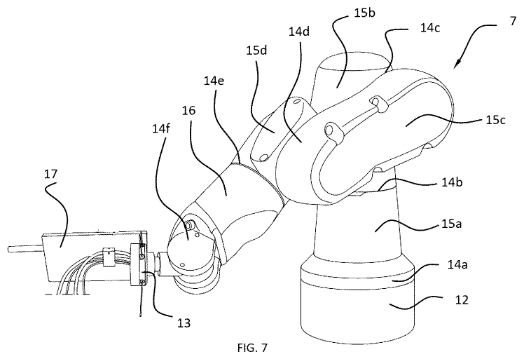

The robotic arm (7) for handling liquid media from/to a recipient according to an embodiment of the invention is illustrated in FIG. 7. The robotic arm shown comprises a base (12), a pipetting tool, and a nozzle tool (13) configured to handle one or more liquid media. The nozzle tool (13) is positioned at a distal end of the robotic arm (7) and has nozzles (17).

In what follows, the robotic arm (7) is described as being mounted on a horizontal surface. Other modes of installation are of course possible, and the adaptation of what follows to such other modes of installation, fall within the scope of the skilled person's abilities, and are considered to form part of the scope of the invention. For instance, the robotic arm (7) can be mounted on a vertical surface, resulting in a rotation of 90° for all subsequent orientations.

The robotic arm (7) is an articulated robotic arm (7) comprising joints (14) and wherein the robotic arm 1 is manufactured by sequentially connecting these joint (14) by multiple links (15). The robotic arm (7) as shown in FIG. 7 comprises six joints (14), allowing movement in six degrees of freedom. More specifically, in a first joint (14a), a base and a proximal end portion of a first link (15a) are connected so as to be rotatable around an axis extending in the vertical direction. The first joint (14a) is a twisting joint.

In a second joint (14b), a distal end portion of the first link (15a) and a proximal end portion of a second link (15b) are connected so as to be rotatable around an axis extending in the horizontal direction. The second joint (14b) is a revolving joint.

In a third joint (14c), a distal end portion of the second link (15b) and a proximal end portion of a third link (15c) are connected so as to be rotatable around an axis extending in the horizontal direction, in this case, parallel to the axis for the second link (15b). It should be noted that deviations from the horizontality of this third axis are possible, but the third axis will always have at least a horizontal component, it being fully horizontal being the most efficient version. The third joint (14c) is in this case a revolving joint.

In a fourth joint (14d), a distal end portion of the third link (15c) and a proximal end portion of a fourth link (15d) are connected so as to be rotatable around an axis in the longitudinal direction of the fourth link (15d). The fourth joint (14d) is a twisting joint.

In a fifth joint (14e), a distal end portion of the fourth link (15d) and a proximal end portion of a connector (16) are connected so as to be rotatable around an axis orthogonal to the fourth axis. The fifth joint (14e) is a revolving joint but it should be noted that this joint can be easily adapted to a twisting joint or rotational joint.

In a sixth joint (14f), a distal end portion of the connector (16) and a proximal end of the nozzle tool (13) are connected so as to be rotatable in a plane orthogonal to the longitudinal direction the connector (16).

Each of the joints (14) is provided with a drive motor as an example of an actuator for relatively rotating the two members connected by the joint (14). The drive motor is, for example, a servo motor which is servo-controlled via a servo amplifier by a control signal transmitted from the controller. In addition, each of the joints is provided with a rotation angle sensor for detecting the rotation angle of the drive motor and a current sensor for detecting the current of the drive motor.

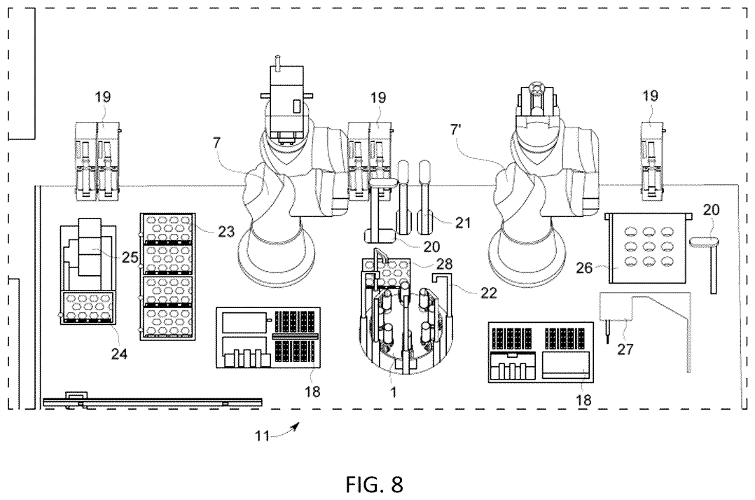

FIG. 8. shows an embodiment of a system (11) comprising a purification device (1) and handling apparatuses: robotic arms (7, 7′), injectors (20, 21, 22, 27), and pumps (19). The robotic arms (7, 7′) make use of needles and/or tips (18). In an embodiment, the compound to be purified is produced upstream in an IVT reaction plate (23). The reaction mixture is heated at 37° C. with a heating unit (25) on a plate (24), prior addition of the enzyme to the reaction plate (23). In an embodiment, the compound of interest obtained using the method disclosed herein is further downstream processed in a container (26).

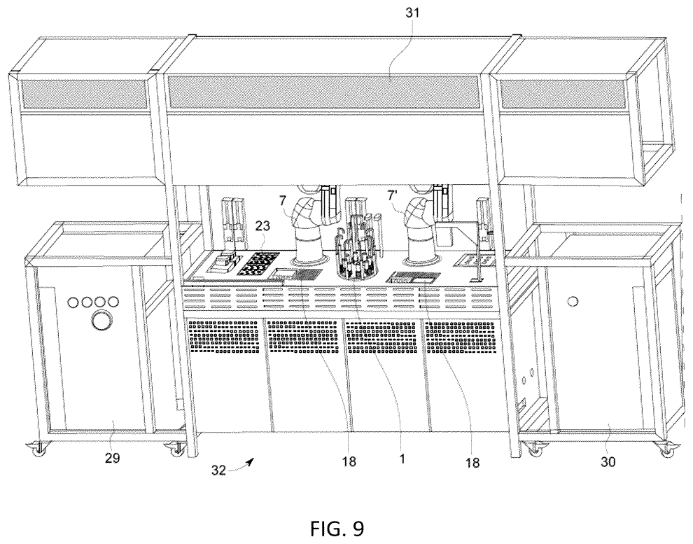

In an embodiment, the system is arranged in a cabinet (32), preferably with a unit for laminar flow generation, as depicted in FIG. 9. The cabinet (32) includes a reactive storage unit (29) that in some embodiments is cooled up to 4° C., a unit for storing the compound of interest after processing (30), and a filter unit (31). The filter unit (31) allows the provision of filtered, sterile air to be circulated within the cabinet (32). Air filtering means may include in some embodiments, a HVAC system with HEPA filters.

### Figure Numbers

1, 1′: device
2: sample plate
3: sample holders
4: base portion of the sample plate
5: magnet unit
6: housing
7, 7′: robotic arm
8, 8′: sample container
9: magnetic particles
10: adaptor
11: system
12: base of the robotic arm
13: nozzle tool
14a-f: joints
15a-e: links
16: connector
17: nozzles
18: needles or tips
19: syringe pumps
20, 21, 22, 27: injectors
23: IVT reaction plate
24 plate
25: heating unit
26: container for downstream processing
28: harvest vessel
29: reagent storage unit
30: compound storage unit
31: filter unit
32: cabinet

1-68. (canceled)

69. A device for separating and/or purifying a compound of interest comprising:
a sample plate, wherein said sample plate comprises a base portion and one or more sample holders positioned on said base portion for receiving one or more sample containers, said one or more sample containers are suitable to hold a liquid sample comprising said compound of interest and a plurality of magnetic particles;
a magnet unit coupled to each sample holder, said magnet unit is configured to capture or to introduce a movement of said plurality of magnetic particles;
wherein said one or more sample holders are configured to perform a mechanical motion, such that said plurality of magnetic particles are mixed within said liquid sample.

70. The device according to claim 69, wherein each sample holder is configured to perform the mechanical motion independently from the remaining sample holders.

71. The device according to claim 69, wherein the mechanical motioning of the sample holders is driven by a motor unit, an electromagnetic unit, or a shaker unit and wherein the mechanical motion can be activated and deactivated.

72. The device according to claim 69, wherein said mechanical motion is a rotation around an axis of said sample holder or a shaking motion.

73. The device according to claim 69, wherein the sample holders are positioned on top of the base portion or are positioned in pockets or recesses of said base portion.

74. The device according to claim 69, wherein said sample plate is configured to rotate around an axis, preferably the central axis of said base portion or is configured to perform a linear movement.

75. The device according to claim 69, wherein said magnet unit is positioned along a side portion of said sample holder or wherein the magnet unit is rod-like and wherein the magnet unit extends at least the entire length of the sample container, preferably extending under the sample container.

76. The device according to claim 69, wherein the magnet unit is arranged in a housing, said housing is open at the side facing the sample holder.

77. A system comprising one or more devices according to claim 1 and at least one handling apparatus configured for dispensing and/or removing a component or a liquid from a sample container present in a sample holder of said device, said handling apparatus comprises an injector or a robotic arm provided with one or more nozzles, needles and/or tips,
and wherein said handling apparatus is connected to one or more reagent storage, waste vessels and/or harvest vessels.

78. The system according to claim 77, comprising a control system arranged and adapted to control the dispensing and/or removing of a component or liquid by said handling apparatus.

79. The system according to claim 77, comprising a plurality of devices according to claim 1, wherein said devices are positioned adjacent to each other and/or said devices are in a nested configuration.

80. The system according to claim 77, further comprising at least one harvest vessel configured to collect a compound of interest.

81. The system according to claim 80, wherein said compound of interest is a nucleic acid, a peptide or a protein preferably DNA or RNA, more preferably mRNA.

82. A method for separating and/or purifying a compound of interest, comprising at least the following steps:
(a) providing one or more sample containers comprising a compound of interest and magnetic particles in a liquid medium, wherein each sample container is positioned in a sample holder;
(b) mixing said compound of interest with said magnetic particles by means of a mechanical motion of said sample holder;
(c) separating said liquid medium from said magnetic particles by capturing or introducing a movement of the magnetic particles towards a magnet unit present in the vicinity of said sample holder;
(d) removing said liquid.

83. The method according to claim 82, wherein after step (d) a liquid is added to said magnetic particles and steps (b) to (d) are repeated.

84. The method according to claim 82, wherein the sample holder with sample container rotates between or after a capturing method step to at least one subsequent position and/or wherein the sample plate rotates between or after a capturing method step, thereby moving the sample holder with a sample container to a subsequent position.

85. The method according to claim 82, wherein in a final step the compound of interest is eluted from said magnetic particles by means of the addition of an elution buffer to said sample container, wherein upon adding said elution buffer, the sample holder is subjected to a mechanical motioning, thereby allowing mixing of said magnetic particles with elution buffer and wherein once the motioning stops, the magnetic particles move towards said magnet unit, thereby causing a separation of the elution buffer comprising the compound of interest and the magnetic particles.

86. The method according to claim 82, wherein said compound of interest is a nucleic acid, a peptide, or a protein preferably DNA or RNA, more preferably mRNA.

87. The method according to claim 82, wherein said method is used for purifying an mRNA molecule from an IVT process, or for a purification step during pre- and/or post-capping.

88. The method according to claim 82, wherein said method is executed by means of a device according to claim 69 or a system according to claim 77.
